(19) United States
(12) Patent Application Publication
(10) Pub. No.: US 2025/0127437 A1
(43) Pub. Date: Apr. 24, 2025

(54) SYSTEM AND METHOD FOR MEASURING CONCENTRATION OF COMPONENT INCLUDED IN BODY FLUID

(71) Applicant: ATONARP INC., Minato-ku, Tokyo (JP)

(72) Inventor: Akihiko NAKAJIMA, Minato-ku, Tokyo (JP)

(73) Assignee: ATONARP INC., Minato-ku, Tokyo (JP)

(21) Appl. No.: 18/833,762

(22) PCT Filed: Jan. 26, 2023
(86) PCT No.: PCT/JP2023/002402

§ 371 Date: Jul. 26, 2024

(30) Foreign Application Priority Data
Jan. 28, 2022 (JP) 2022-011549

(51) Int. Cl. A61B 5/1455; A61B 5/00; G01J 3/44

(52) U.S. Cl. CPC A61B 5/1455; A61B 5/6843; G01J 3/44

(57) ABSTRACT

A system for measuring a concentration of a component included in a body fluid includes: an apparatus that acquires data including spectra in a time series obtained by irradiating at least part of the body fluid in a flowing state with laser light; and an analyzer apparatus that analyzes analysis target spectrum included in the acquired data based on an analysis reference spectrum that is highly similar to the analysis target spectrum, out of a plurality of reference spectra that primarily reflect one out of a plurality of principal components of the body fluid, and determines the concentration of a target component in the body fluid.

### TECHNICAL FIELD

The present invention relates to a system and method for measuring the concentration of a component included in a body fluid of a living organism.

### BACKGROUND ART

International Publication WO2019/117177 discloses the provision of a distinguishing method, a learning method, a distinguishing apparatus, and a computer program that make it possible to distinguish between types of cells more accurately than previously based on Raman spectra obtained from cells. In this method of distinguishing between types of cell included in a sample, one Raman spectrum is obtained from one unidentified cell, and a plurality of match values indicating the degree of match between the Raman spectrum of the unidentified cell and a plurality of principal component spectra obtained by principal component analysis of a plurality of Raman spectra, which are composed of Raman spectra that have been individually obtained from a plurality of cells whose type has been distinguished, are calculated. The type of the unidentified cell is determined by using a learning model that uses supervised learning in which the plurality of match values is classified according to type based on the result of classifying a plurality of principal component scores, which correspond to each of the plurality of cells of known types obtained by the principal component analysis.

Japanese Laid-open Patent Publication No. 2012-27008 discloses the measurement of plasma glucose via the steps below to provide a technology that measures plasma glucose concentration with improved accuracy. A sample preparation step of hemolyzing blood cells within the blood to prepare a measurement sample, a whole blood glucose measuring step of measuring the glucose concentration of whole blood using this measurement sample, and a whole blood liquid component ratio calculation step of calculating the ratio of liquid components in whole blood from the ratio of blood cells to plasma in the blood and the known values of the liquid component ratio of blood cells and the liquid component ratio of plasma are described.

### SUMMARY OF INVENTION

A body fluid of a living organism, such as blood, includes blood cells and plasma as its main components (principal components). The expression “blood cells” includes red blood cells, white blood cells, platelets, and the like. Apart from the blood's principal components, measurement of substances (components) such as glucose, hemoglobin A1c, creatinine, and albumin in blood may be required to check the health of the living organism. Plasma blood glucose level may be required as the measurement standard for blood glucose level. It is not easy to accurately measure the concentration of a target component in a body fluid such as blood, which contains many components. In addition, there are cases where the measured value in a specific constituent component (one of the principal components) of a body fluid, such as plasma glucose level, is important as an indicator.

One aspect of the present invention is a system for measuring a concentration of one or more components included in a body fluid. This system includes: an apparatus that is configured to acquire data including spectra (a plurality of spectra) in a time series (temporal sequence) obtained by irradiating at least a part of the body fluid in a flowing state with one or more laser lights; and an analyzer apparatus that is configured to analyze an analysis target spectrum, which is included in the acquired data, based on an analysis reference spectrum, which is highly similar to the analysis target spectrum and is selected out of a plurality of reference spectra that primarily reflect one out of a plurality of principal components of the body fluid respectively, and determine the concentration of a target component in the body fluid.

Attempts have been made to obtain the glucose concentration in blood from a Raman spectrum obtained by irradiating a body fluid, such as blood, with laser light. One example of an obtaining method under consideration follows the conventional procedure for measuring the glucose concentration in whole blood, and the method includes averaging a plurality of Raman spectra obtained in a time series and determining the glucose concentration from the height or shape of glucose-related peaks included in an averaged spectrum. However, the inventors of the present application found that when Raman spectra are obtained from a body fluid in a flowing state, such as blood flowing through a blood vessel, at intervals that are shorter than the flow rate of blood cells, the acquired Raman spectra will not provide information on average concentrations and the acquired spectra will change over time, especially in a comparatively narrow blood vessel.

It may be possible to average such spectra to obtain a spectrum for whole blood. However, if spectra that change over time contain information about blood cells and plasma, which are the principal components of blood, as well as information about different components such as red blood cells, white blood cells, and platelets, averaging a plurality of spectra that reflect a number of different principal components can result in information indicating the concentration of a target component, such as information about the concentration of glucose, which is typically smaller than the information on the principal components becoming buried. Accordingly, simply averaging a large number of spectra does not improve measurement accuracy, and may conversely make it difficult to measure the concentration of a target component with high accuracy.

In addition, the inventors of the present application found that for data containing a plurality of spectra in a time series obtained by irradiating a body fluid in a flowing state with laser light, it is possible to classify (distinguish, resolve, and fractionate) this plurality of spectra in a time series, and that these spectra include fractionated (time-divided) information on the principal components of the body fluid. In other words, by acquiring spectra of a body fluid, in which the principal components are not uniform, in a state where the body fluid is flowing, it is possible to obtain information in which these principal components have been fractionated or reflected dividedly. For this reason, by analyzing an analysis target spectrum based on (as a reference) an analysis reference spectrum which, out of a plurality of reference spectra that primarily reflect one of the plurality of principal components of the body fluid, is highly similar to the analysis target spectrum, it is possible to measure the concentration of the target component included in the body fluid with high accuracy. In addition, it is possible to identify one of the plurality of principal components and determine the concentration of the target component in that principal component.

For example, in the case of blood, the plurality of reference spectra include a spectrum with blood cell components as the principal component, a spectrum with plasma components as the principal component, and a spectrum with a mixture of such components. A reference spectrum with blood cell components as a principal component may be further divided into a spectrum with red blood cell components or the like as the principal component. By performing analysis of an analysis target spectrum, which is similar to a spectrum including plasma as a principal component based on the average spectrum including a plasma component, out of the plurality of reference spectra as the reference spectrum, it is possible to prevent information of other principal components, such as blood cell components, from being disadvantageous (that is, acting as noise) when used as reference information during analysis, and thereby improve the measurement accuracy of the concentration of a target component, such as glucose in or with the plasma component. In addition, it is also possible to directly acquire the plasma glucose concentration, which should be a reference value for determining diabetes or the like.

Reference spectra that mainly contain components such as blood cell components and plasma components respectively may be provided in advance as spectra that include information on these respective components. The analysis reference spectrum may be determined based on a group of similar spectra that include highly similar spectra that are included in the data obtained in a time series and repeatedly appear in the plurality of spectra. In addition, a reference spectrum may be obtained from the group or groups of similar spectra and a plurality of standard (normal) spectra in which any of (one of) a plurality of principal components of the body fluid is dominant, respectively. The system may also include an apparatus that generates the reference spectrum, or an apparatus that automatically generates (self-learns) a reference spectrum from highly similar spectra.

The apparatus that acquires the data may include an apparatus that acquires the data from the cloud or the like, or may be a detection apparatus that acquires the data onsite from a living organism. The detection apparatus may include a Raman spectrometer that acquires a Raman spectrum and may be a device that non-invasively acquires the data from a living organism. The body fluid is typically blood, and the plurality of principal components may include a plasma component and a blood cell component. The plurality of principal components may include at least one of red blood cells, white blood cells, and platelets, and a plasma component. The target component may include at least one of glucose, hemoglobin A1c, creatinine, and albumin.

Another aspect of the present invention is a method of detecting a component in a body fluid. This method includes: acquiring data including a plurality of spectra in a time series obtained by irradiating a body fluid in a flowing state with laser light; analyzing an analysis target spectrum, which is included in the acquired data, based on an analysis reference spectrum, which is highly similar to the analysis target spectrum and is selected out of a plurality of reference spectra that primarily reflect one out of a plurality of principal components of the body fluid respectively; and determining a concentration of a target component in the body fluid. This method may further include determining the analysis reference spectrum based on a group of highly similar spectra that repeatedly appear among the plurality of spectra included in the acquired data.

Yet another method of measuring a concentration of a component included in a body fluid. The method includes: acquiring data including a plurality of spectra in a time series obtained by irradiating at least a part of the body fluid in a flowing state with laser light; analyzing an analysis target spectrum, which is included in a group of similar spectra including highly similar spectra that repeatedly appear in the plurality of spectra included in the acquired data, based on an analysis reference spectrum including spectral components that are common to the group of similar spectra; and determining the concentration of a target component in the body fluid.

The determining may include determining the concentration of the target component, which is included in any one of the plurality of principal components of the body fluid. The acquiring may include acquiring the data from a living organism, the acquiring may include acquiring a Raman spectrum, and the data may be acquired by these methods non-invasively.

### BRIEF DESCRIPTION OF DRAWINGS

FIG. 1 depicts one example of a system that acquires a CARS spectrum for blood flowing in a blood vessel.

FIG. 2 depicts one example of a probe.

FIGS. 3(a) and 3(b) depict one example of CARS spectra of blood.

FIGS. 4(a) and 4(b) depict examples of CARS spectra of whole blood.

FIGS. 5(a), 5(b), and 5(c) depict examples of CARS spectra obtained from a living organism (human body).

FIGS. 6(a), 6(b), 6(c), and 6(d) depict examples of CARS spectra of blood flowing through a blood vessel.

FIGS. 7(a) and 7(b) depict examples of CARS spectra with red blood cells as a principal component.

FIGS. 8(a) and 8(b) depict examples of CARS spectra with a plasma component as a principal component.

FIGS. 9(a) and 9(b) depict examples of CARS spectra for blood that reflect glucose concentration.

FIG. 10 depicts the correlation between glucose concentration and relevant peak intensities in RBC-like CARS spectra.

FIG. 11 depicts the correlation between glucose concentration and related peak intensities in plasma-like CARS spectra.

FIG. 12 is a flowchart depicting one example of a method of measuring glucose concentration.

### DESCRIPTION OF EMBODIMENTS

FIG. 1 depicts an overview of a system (body fluid inspection system, biological monitoring system, or blood testing system) according to the present invention that observes (monitors) the blood flowing through a blood vessel of a sample (which may be a living organism) and acquires a Raman spectrum derived from blood. One example of such a system is a biological (living body) monitoring system 30 that irradiates a blood vessel 5a of a living organism 5 with laser lights, obtains a CARS (Coherent Anti-Stokes Raman Scattering) spectrum 51 with blood flowing through the blood vessel 5a as the monitoring (detection) target 5t, and monitors the condition of the living organism 5. This biological monitoring system 30 may include a medication system (dosing system) 38 that administers (injects) one or more drugs to maintain the health of the living organism 5. One example of the biological monitoring system 30 is a wearable mobile terminal, such as a smartwatch, with a built-in communication function and user interface. One example of the medication system 38 is a system that administers one or more drugs via the skin of the living organism 5, and may include an injector 38a and a supply apparatus (supply unit) 38b that supplies a predetermined drug to the injector 38a.

One example of a biological monitoring system (biological management system) 30 is a measurement system (blood glucose measurement apparatus) that measures a blood glucose level. The biological monitoring system 30 includes a detection apparatus 31 including a Raman spectrometry apparatus (optical system) 10 that acquires CARS spectra 51 from the blood 5t flowing through the blood vessel 5a, and a blood glucose monitor 33 that analyzes the CARS spectra 51 that have been obtained from the detection apparatus 31 via an input interface 32 and outputs a plasma blood glucose level. The Raman spectrometry apparatus 10 depicted in FIG. 1 is one such example, and depicts a system in which a mouse's ear is used as the living organism 5 for experimental purposes, and the blood 5t flowing through the blood vessel 5a in the ear is monitored. The Raman spectrometry apparatus 10 is not limited to this example, and may be any device that can obtain signals from the blood 5t flowing in the blood vessel 5a non-invasively via the skin of the living organism 5. In addition, the Raman spectrometry apparatus 10 may use a minimally invasive method, such as controlling the optical path with an implant embedded into the living organism, or implanting an artificial blood vessel (bioport) into the living organism to form a blood flow just below the skin.

The Raman spectrometry apparatus 10 includes a probe (probe end or sampler) 13 for irradiating the blood 5t in the blood vessel being monitored with laser lights to obtain CARS light, a laser source 11 for irradiating the blood 5t with pump light (with a wavelength of 1030 nm, for example) 59p and Stokes light (with a wavelength of 1100 to 1300 nm, for example) 59s via the probe 13, and a spectrometer 12 that obtains a spectrum (spectra) 51 of the CARS light 50 emitted from the blood 5t. The Stokes light 59s may be broadband light with a wide wavelength band, or narrowband light produced by a tunable laser. The laser source 11 may additionally output probe light (with a wavelength of 780 nm, for example), and the Raman spectrometry apparatus 10 may acquire time-dependent CARS spectra for which a delay time has been taken into account.

This Raman spectrometry apparatus 10 is suitably configured for use in experiments, and further includes a visible light source 15 that enables the position of the laser irradiated onto the sample 5 via the probe 13 to be optically confirmed, a camera (CCD camera) 16 for confirming the position of the laser using visible light that has been transmitted through the sample 5, and dichroic mirrors 17 and 18 for separating the laser and CARS light from the visible light. One example of the dichroic mirror 17 for separating laser light and visible light is a filter that transmits wavelengths of 750 nm and above, and one example of the dichroic mirror 18 for separating the CARS light and visible light is an SP filter centered on a wavelength of 805 nm. The Raman spectrometry apparatus 10 may include optical elements that form appropriate optical paths, such as mirrors M1 to M4 and prisms.

An fS (femtosecond) to pS (picosecond)-pulsed laser is emitted from the laser source 11, and the spectrometer 12 intermittently (in a time series, in a temporal sequence) obtains an analysis target spectrum (first spectrum) 51 that is integrated in units of 8 mS (milliseconds), for example. Accordingly, the detection apparatus 31 can output data 52 including a plurality of analysis target spectra 51 that were obtained in a time series, and the blood glucose monitor 33 can acquire this data 52 via the input interface 32. Note that this pulse width and integration time are mere examples.

The blood glucose monitor 33 includes an analyzer apparatus 20 that is configured to analyze the analysis target spectrum 51 included in the data 52 that has been acquired via interface 32. The analyzer apparatus 20 includes a first analysis unit 21 that analyzes the analysis target spectrum 51, which reflects the components in a body fluid and is obtained by irradiating the body fluid with lasers, based on an analysis reference spectrum 53 which, out of a plurality of reference spectra (second spectra) 54, each of which mainly reflects one of a plurality of principal components of the body fluid, is highly similar to the analysis target spectrum 51, and determines the concentration of a target component, for example, a substance such as glucose, included in the body fluid. The first analysis unit 21 may be an analysis unit that determines the concentration using an analysis technique such as multivariate analysis (principal component analysis) based on the analysis reference spectrum 53. The first analysis unit 21 may include a learning model (AI(1)) 21a that has been trained in advance to estimate the concentration of one or more specified target components based on an analysis reference spectrum 53 that is highly similar to the analysis target spectrum 51.

The analyzer apparatus 20 may further include a reference spectrum generating apparatus 22 that is configured to determine the analysis reference spectrum 53 based on a group of similar spectra 55 including highly similar spectra that repeatedly appear in the plurality of spectra 51 included in the acquired data 52. The generating apparatus 22 may include a device (AI(2)) 22a that self-learns a plurality of reference spectra (second spectra) 52 from groups 55 of a plurality of spectra, out of the plurality of analysis target spectra (first spectra) 51, in which some spectral components exhibit high correlation or similarity.

The analyzer apparatus 20 uses the learning model 21a to obtain the concentration of glucose (target) component included in the blood 5t from the plurality of analysis target spectra 51 that were obtained in a time series (that is, intermittently over time) by irradiating laser light onto a body fluid (here, blood) 5t in a flowing state using the Raman spectrometry apparatus 10. The learning model 21a may be trained to obtain the concentration of a target component, such as glucose, based on a plurality of reference spectra (second spectra) 54 that are provided in advance and have been stored in a library 25. The self-learning device 22a may refer to a standard (normal) spectrum 56 of each of the principal components of blood, such as red blood cells and plasma, that has been obtained in advance, and generate (self-learn) reference spectra or spectrum 54, which include the analysis reference spectrum 53 that serves as a standard for determining concentrations, from groups 55 of a plurality of spectra, out of the plurality of analysis target spectra 51 obtained from the Raman spectrometry apparatus 10, that have high similarity or correlation with the standard spectrum 56.

The analyzer apparatus 20 may include a function which, or be configured to, through cooperation between the learning models 21a and 22a, analyze an analysis target spectrum 51, which is included in a group of similar spectra 55 that include highly similar spectra that repeatedly appear in the plurality of spectra 51 included in the acquired data 52, based on an analysis reference spectrum 53 including spectral components that are common to the group of similar spectra, and determine the concentration of a target component in the body fluid.

The biological monitoring system 30 may include an output interface 35 that is configured to output the obtained concentration of the target component, such as glucose. The output interface 35 may output the measured value as a concentration of glucose in blood, and may include a function (plasma glucose output device) 35a that refers to principal components, such as plasma, included in the analysis reference spectrum 53 that is referred to when measuring the concentration and output the value as a concentration of glucose in (with) the principal components that flows together with (in this case “the plasma glucose level”). The output interface 35 may also include a function 35b for outputting, for each component, the concentration of a target component contained in other principal components, such as red blood cells, without being limited to plasma.

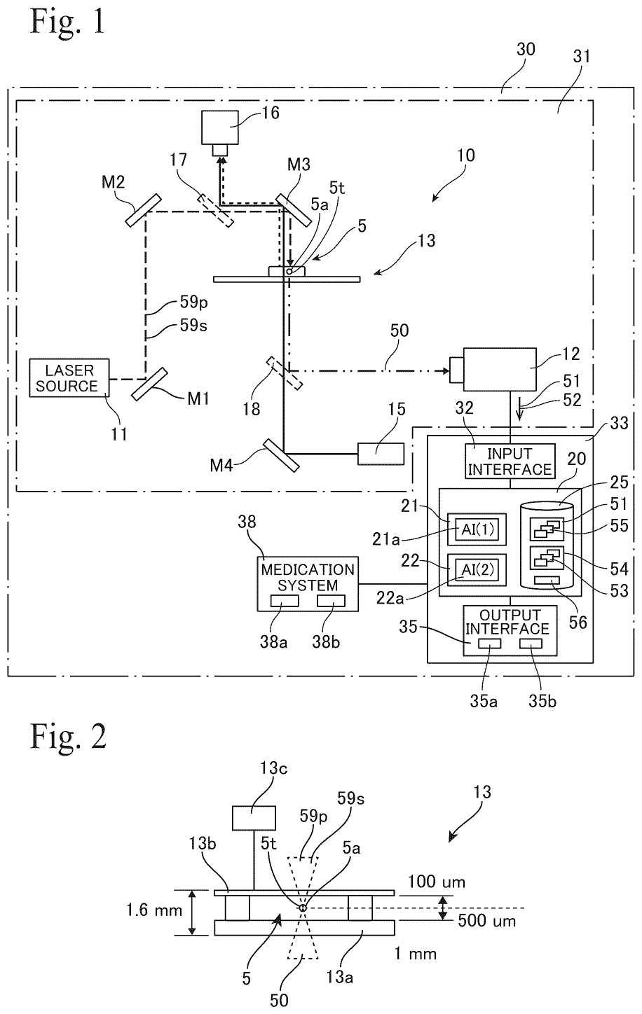

FIG. 2 depicts one example of the probe 13. This probe 13 clamps the earlobe 5 of a mouse, which is the sample of this system 30, and irradiates the blood 5t flowing through a blood vessel 5a in the earlobe 5 with laser lights 59p and 59s to acquire the CARS light 50 non-invasively. The probe 13 includes upper and lower translucent plates 13a and 13b that clamp the earlobe 5, and an actuator, such as a piezoelectric actuator 13c, that can change the distance between the plates 13a and 13b. The actuator 13c of the probe 13 functions as a mechanism that compresses the blood vessel 5a to control the flow rate of the blood 5t.

By compressing the blood vessel 5a, it is possible to control the cross-sectional area of the blood vessel 5a and thereby control the flow rate of the blood 5t flowing through the blood vessel 5a. As one example, by reducing the cross-sectional area, it is possible to slow the blood flow and resist (slow down) the passage of blood cell components, such as red blood cells, which are a principal component of the blood 5t, thereby making it easier to create a time period during which blood cell components (red blood cells) pass only intermittently (not continuously) and between that the plasma component is predominant. Accordingly, by measuring the blood 5t flowing through the blood vessel 5a, it is possible to measure the principal components contained in the blood 5t, such as red blood cells and plasma, according to time division, and spectra in which the principal components are more characteristically represented can be obtained. The probe 13 may be in the form of a finger clip or other type that clamps a thin portion of the skin, or may be a type that is pressed against the skin to apply pressure to capillaries at the surface of the skin. The probe 13 may acquire CARS light 50 that has passed through the skin in the forward direction relative to the incident laser lights 59p and 59s, or may acquire CARS light 50 that has been emitted backward or obliquely relative to the incident laser lights 59p and 59s. It should be noted that although the size depicted in FIG. 2 is suited to clamping onto and applying pressure to the earlobe of a mouse as the sample 5, the size of the probe 13 is not limited to this.

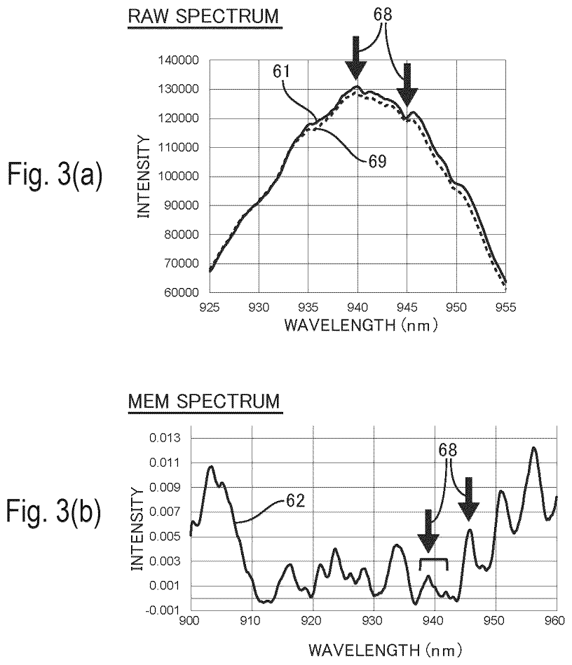

FIG. 3 depicts one example of a CARS spectrum of the blood 5t (in-vitro, ex-vivo). FIG. 3(a) depicts the CARS spectrum (raw spectrum) 61 of a sample (whole blood) that has been hemolyzed after collection of the blood 5t from a mouse, compared with the CARS spectrum 69 of water. The CARS spectrum 61 of the hemolyzed sample exhibits clear differences from the CARS spectrum 69 of water in some regions 68. FIG. 3(b) depicts a spectrum (MEM spectrum) 62 obtained by analyzing the CARS spectrum of a hemolyzed whole blood sample according to the maximum entropy method (MEM). In the MEM spectrum 62, the features of the original spectrum 61 are emphasized.

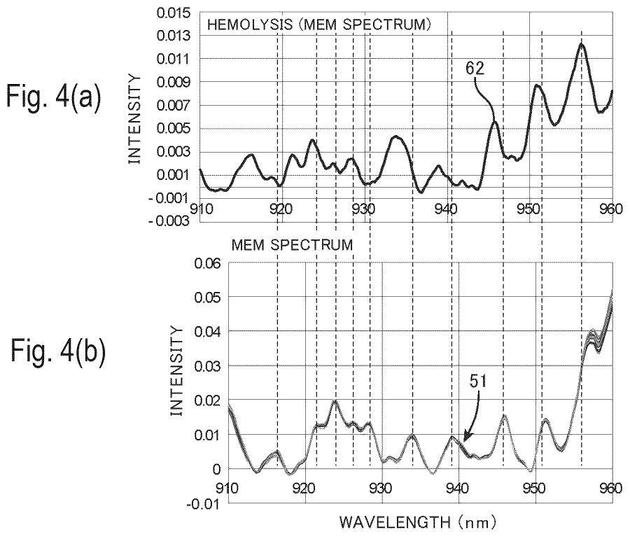

FIG. 4 depicts one example of a CARS spectrum 51 obtained by the system 30 described above from the blood 5t (in vivo) flowing through a blood vessel 5a in the ear 5 of a mouse. FIG. 4(a) depicts the CARS spectrum (MEM spectrum) 62 of whole blood (hemolyzed blood) in FIG. 3(b) for comparison purposes, and FIG. 4(b) depicts a MEM spectrum 51 obtained by the system 30. These in-vivo CARS spectra 51 have similar features to the in-vitro MEM spectrum 62 of the hemolyzed sample depicted in FIG. 3(b). Accordingly, it can be understood that the system 30 can noninvasively and accurately obtain CARS spectrum 51 indicating the blood 5t that flows through the blood vessel 5a.

Note that the MEM spectra 51 depicted in FIG. 4(b) indicate the results of measurements for one second with an integration time of 25 msec (milliseconds), and indicates an overview of 40 spectra obtained in one second.

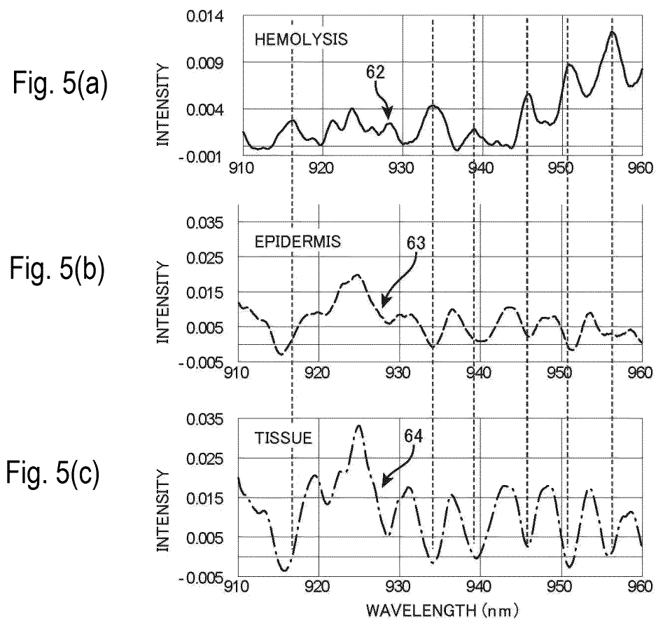

FIG. 5 shows a comparison between several examples of CARS spectra obtained from a living organism. FIG. 5(a) depicts a CARS spectrum (MEM spectrum) 62 of hemolyzed blood (whole blood). FIG. 5(b) depicts an example of a CARS spectrum (MEM spectrum) 63 of shallow skin. FIG. 5(c) depicts one example of a CARS spectrum (MEM spectrum) 64 of tissue under the skin with any blood vessels removed. Each spectrum has different features, and it can be understood that the system 30 makes it possible to distinguish a CARS spectrum that originates from a blood vessel for cases where a blood vessel 5a has been set as the observation target. Using the camera 16, the system 30 also makes it possible to confirm, by means of images, the position at which the laser is being irradiated to obtain the CARS spectrum.

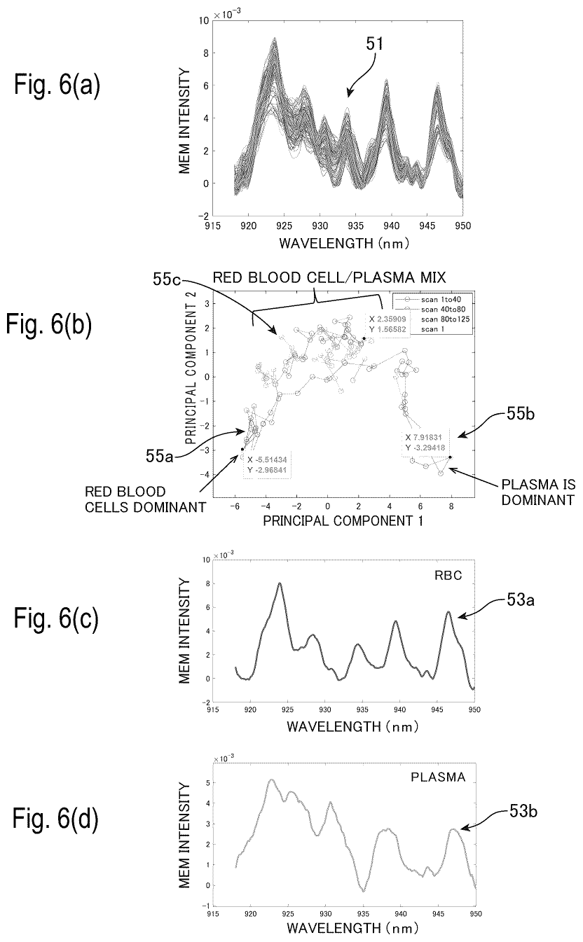

FIG. 6 depicts more detailed analysis of a CARS spectrum (MEM spectrum, in-vivo) 51 obtained by the present system 30 from the blood 5t flowing through a blood vessel 5a. FIG. 6(a) depicts CARS spectra (MEM spectra) 51 that have been integrated in 8 msec (millisecond) units and indicates a plurality of spectra 51 which were (intermittently) obtained in a time series during a one-second period and shown overlayed. These CARS spectra 51 are examples of spectra included in the acquired data 52 used in the system 30 depicted in FIG. 1. These spectra 51 are CARS spectra obtained from the blood 5t, and although the overall trends appear to be similar, it can be understood that several patterns of spectra with different peak heights or the like repeatedly appear.

FIG. 6(b) depicts the results of principal component analysis (PCA) performed on these spectra 51. Principal component analysis, which is one type of multivariate analysis, makes it possible to synthesize a small number of principal component variables that are uncorrelated and best represent the overall variance from a large number of correlated variables, thereby reducing the dimensionality of the data. In the present embodiment, the spectra 51 acquired from blood can be classified into three highly similar groups 55a, 55b, and 55c. The first group 55a is a group of spectra that mainly reflect components of blood cells, and in particular red blood cells (RBCs), the second group 55b is a group of spectra that mainly reflect a plasma component, and the third group 55c is a group of spectra in which red blood cells and plasma are mixed.

FIG. 6(c) depicts a representative spectrum for the first group (spectra group) 55a, as one example, a spectrum 53a obtained by averaging the spectra in the group 55a. This spectrum (RBC spectrum) 53a is assumed to be a spectrum that strongly reflects a blood cell, and in particular, a red blood cell (RBC) component. FIG. 6(d) depicts a representative spectrum for the second group (spectra group) 55b, as one example, a spectrum (plasma spectrum) 53b obtained by averaging the spectra in the group 55b. This spectrum 53b is assumed to be a spectrum that strongly reflects a plasma component. In this way, it can be understood that the CARS spectra 51 obtained from the blood 5t flowing through a blood vessel 5a contains three broad patterns of spectra that repeat over time, that is, spectra belonging to the group 55a of similar spectra that mainly reflect the red blood cell component, spectra belonging to the group 55b of similar spectra that mainly reflect the plasma component, and spectra belonging to the group 55c of similar spectra that reflect both red blood cell and plasma components.

From analysis by the present inventors, it has been determined that the time interval at which the spectra belonging to the spectra groups 55a to 55c that have different patterns are repeated will depend on the flow rate of the blood. The flow rate of blood 5t in blood vessel 5a will change according to variations in the diameter and shape of the blood vessel due to the applied pressure compressing the blood vessel 5a, or when the amount of blood glucose changes, such as after a meal, and it was found that fluctuations (tendencies) in the flow rate of blood are significantly correlated to fluctuations (tendencies) in the pattern of the CARS spectra 51 obtained from the blood. In addition, by further slowing the speed (blood flow) of the blood 5t flowing through the blood vessel 5a and/or shortening the integration time of the CARS spectra 51 obtained from the flowing blood 5t, it is considered possible to identify spectra that strongly reflect other main components of the blood, such as platelets and white blood cells, as patterns that may repeat.

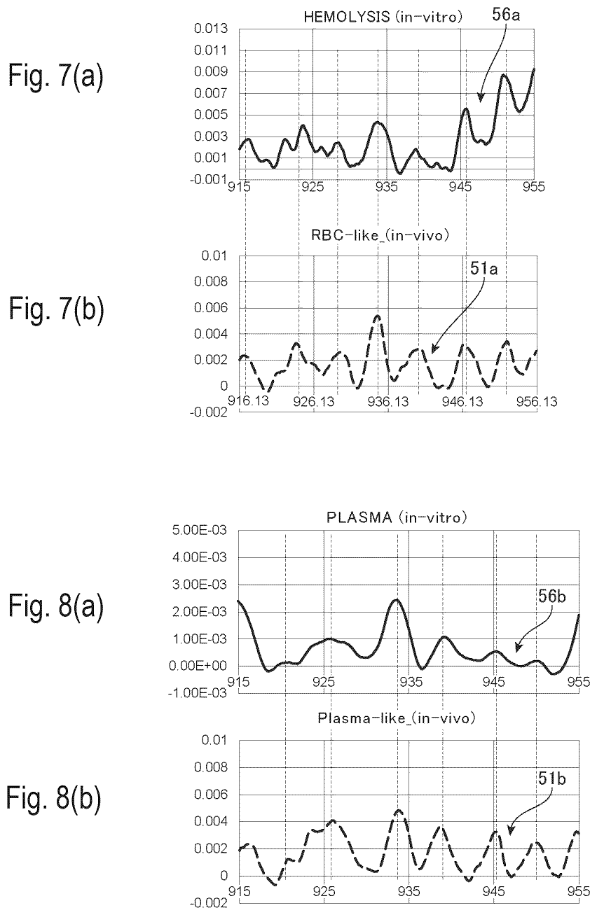

FIG. 7 depicts a comparison between an example of a CARS spectrum 56a (see FIG. 7(a)) obtained from a sample in which red blood cells were hemolyzed outside the body (in-vitro) and an example of a CARS spectrum 51a (see FIG. 7(b)) obtained non-invasively (in-vivo) by the system 30 at timing which is believed to reflect a red blood cell component. It is believed that these spectra will have common features.

FIG. 8 depicts one example of a CARS spectrum 56a (see FIG. 8(a)) obtained from a plasma sample outside the body (in-vitro), and one example of a CARS spectrum 51b (FIG. 8(b)) obtained non-invasively (in-vivo) by the system 30, at a timing which is believed to reflect a plasma component. It is believed that these spectra will also have common features. Accordingly, by observing the blood 5t flowing through the blood vessel 5a and obtaining a plurality of CARS spectra 51 continuously in a time series or intermittently by obtaining the result of averaging or integrating over a sufficiently short period of time, it can be understood that the CARS spectra 51b and 51a, which respectively reflect different components out of the principal components of the blood 5t, such as plasma and blood cells (in the present embodiment, red blood cells), can be obtained at a predetermined timing in keeping with the blood flow. These results are believed to be particularly evident when observing blood flowing through capillaries or similarly narrow blood vessels close to the surface of the skin.

FIG. 9(a) depicts a plurality of CARS spectra 51 obtained by the system 30 according to the present embodiment in one second after glucose solution has been injected into the blood of the mouse 5. These CARS spectra 51 are believed to reflect a change in the glucose concentration in the blood 5t. In addition, out of such CARS spectra 51, CARS spectra 51a that have been determined to belong to the group 55a with red blood cells as the principal component have been extracted and depicted in FIG. 9(b). These spectra (RBC-like spectra) 51a that mainly reflect the red blood cell component appear periodically in the plurality of CARS spectra 51 acquired in a time series (that is, over time) in the data 52. Accordingly, the RBC-like spectra 51a may be selected out of the CARS spectra 51 included in the data 52 based on time (or timing or time intervals), or spectra that have been determined to be similar to the RBC spectrum 53a may be selected. The CARS spectra (plasma-like spectra) 51b determined to belong to the group 55b whose principal component is plasma can also be selected (extracted) in the same way from the plurality of CARS spectra 51 included in the data 2. The plasma-like spectra 51b may be selected out of the CARS spectra 51 included in the data 52 based on time (or timing or time intervals), or spectra that have been determined to be similar to the plasma spectrum 53b may be selected.

In addition to variations due to differences in the principal component, the CARS spectra 51 depicted in FIG. 9(a) also exhibit variations due to glucose concentration. As one example, according to preliminary analysis by the present inventors, it was found that glucose concentration is strongly reflected in values near a wavelength of 928 nm (indicated as “Pg1”), while values in the vicinity of wavelengths of 926 to 927 nm (indicated as “Pg0”) are less affected by the glucose concentration. Accordingly, by comparing the spectra 51a classified as RBC-like with standard RBC spectra 53a, also comparing the spectra 51b classified as plasma-like with plasma spectra 53b, respectively, it is possible to set glucose as the target for each of the different principal component peaks and analyze a relationship that indicates glucose concentration individually with greater precision. As described earlier, one suitable method is to employ a learning model AI 21 that has previously learned how glucose concentration changes based on the respective spectra 53a and 53b, respectively.

One example of a simple method for determining glucose concentration is to compare spectra 51a that have been classified as RBC-like with the standard RBC spectrum 53a, rescale (that is, enlarge or reduce) the spectra so that the intensities (values) at wavelengths of 920 to 930 nm, for example, are the same, and make the determination by obtaining the correlation between the intensities (values) between specified peaks and the glucose concentration in advance. As one example, for RBC-like spectra 51a, from analysis by the present inventors, it was found that there is a high correlation between the difference (I₁−I₀) between the intensity I₁ at a wavelength of 928 nm and the intensity I₀ at a wavelength of 926.2 nm and the glucose concentration, and that the glucose concentration in blood, and in particular the glucose concentration that moves together with red blood cells, can be determined with high accuracy. For the plasma-like spectra 51b, it was found that there is a high correlation between the difference (I₃−I₂) between the intensity I₃ at a wavelength of 928.5 nm and the average intensity I₂ at a wavelength of 926 to 927 nm and the glucose concentration, and that the glucose concentration in the blood, and in particular, the glucose concentration that moves together with plasma (plasma blood glucose concentration) can be determined with high accuracy.

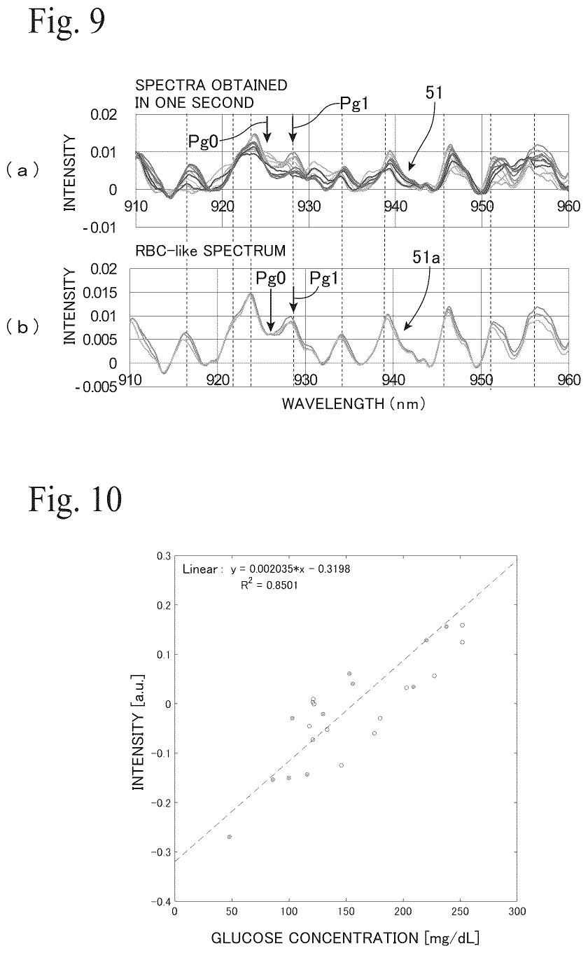

FIG. 10 depicts the correlation between the differences in intensity of the glucose-related peaks included in the RBC-like spectra 51a selected in FIG. 9(b), that is, the difference (I₁−I₀) mentioned above in the present embodiment, and glucose concentrations obtained by a glucose meter (Glucometer (SMBG), “Freestyle Freedom Lite” provided by Nipro Corporation). As shown in this figure, in the analyzer apparatus 20, an RBC-like spectrum (the analysis target spectrum, first spectrum) 51a included in the RBC-like group 55a among the plurality of CARS spectra 51 obtained from the blood 5t flowing through a blood vessel 5a can be analyzed by selecting an RBC spectrum 53a (from the plurality of CARS spectra 51) that should be the standard as an analysis reference spectrum (second spectrum), and the concentration of glucose, which is the target, in the blood, can be determined (estimated) as the glucose concentration in the blood, in particular the glucose concentration flowing together with the red blood cells (that is, in the red blood cells) with extremely high accuracy.

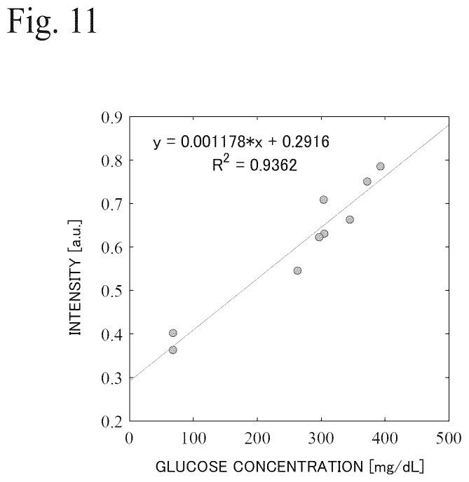

FIG. 11 depicts the result of calculating the concentration of glucose, which is the target, in the blood, by using a target plasma spectrum 51b in the same way, that is the plasma-like spectrum (analysis target spectrum, first spectrum) 51b included in the group 55b of CARS spectra (plasma-like spectra) that have plasma as a principal component out of the CARS spectra 51 included in the data 52 can be analyzed by selecting the plasma spectrum 53b that should be the standard as an analysis reference spectrum (second spectrum) and the concentration of glucose, which is the target, in the blood can be calculated. In more detail, FIG. 11 depicts the correlation between the differences in intensities between glucose-related peaks included in the plasma-like spectra 51b, that is, the differences (I₃−I₂) mentioned above in the present embodiment, and glucose concentrations obtained by the glucose meter.

As shown in FIG. 11, there is a high correlation between the glucose concentration (intensity) that is determined from the plasma-like spectra 51b obtained by time-division (fractionating, selecting in time) the CARS spectra 51 obtained from the blood 5t, with reference to a standard or averaged plasma spectrum 53b of the plasma-like spectra 51b, and the glucose concentration obtained by the glucose meter. Accordingly, the concentration in blood of glucose that is the target can be measured with extremely high accuracy. Additionally, the concentration (in plasma) of glucose that flows through a blood vessel together with the plasma can be determined (estimated). Plasma glucose concentration is referred to as a monitoring value for blood glucose in diabetics, and some glucose meters are designed to display the plasma glucose concentration by correcting for hematocrit. In contrast, with the system 30 according to the present embodiment, it is possible to derive the plasma glucose concentration directly from the plasma-like CARS spectra 51b.

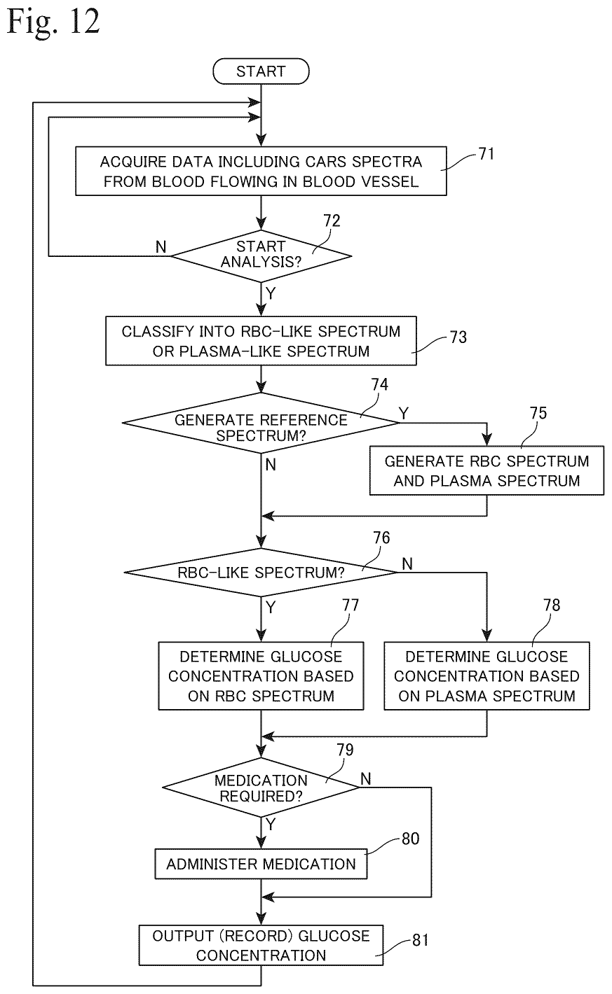

FIG. 12 is a flowchart depicting a method used in or by controlling the system 30 according to the present embodiment to determine the concentration of a target, for example, glucose, from blood flowing through a blood vessel. In step 71, the blood glucose monitor 33 acquires, via the input interface 32, the data 52 that includes a plurality of CARS spectra 51 from the blood 5t flowing through the blood vessel 5a in a time series (temporal sequences). The data 52 may be data acquired on-site or in real time from the detection apparatus 31, or may be data that was measured in the past and stored in advance on the cloud or the like. In step 72, when the input interface 32 or the analyzer apparatus 20 determines that CARS spectra 51 that were measured over a certain period of time have been obtained or that sufficient amount of data 52 for analyzing have been acquired, analysis of the CARS spectra 51 included in the data 52 commences.

The analyzer apparatus 20 analyzes the analysis target spectrum 51 contained in the acquired data 52 based on an analysis reference spectrum which, out of a plurality of reference spectra that mainly reflect one of a plurality of principal components of the body fluid respectively, is highly similar to the analysis target spectrum, and determines the concentration of the target (here, glucose) component in the body fluid (here, blood). First, in step 73, the first analysis unit 21 determines an analysis reference spectrum based on one or more groups of highly similar spectra that repeatedly appear in the plurality of spectra 51 contained in the acquired data 52. In more detail, the CARS spectra 51 included in the data 52 are classified into group 55a of RBC-like spectra, whose principal component is red blood cells and group 55b of plasma-like spectra, whose principal component is plasma, and the RBC spectrum 53a or the plasma spectrum 53b is selected as the analysis reference spectrum to be referred to during analysis.

At this time, the reference spectrum generating apparatus 22 may automatically generate an analysis reference spectrum including spectral components common to a group of similar spectra. In more detail, in step 74, when the auto-generation of a reference spectrum has been set, in step 75, the reference spectrum generating apparatus 22 may extract average components from each of the group of RBC-like spectra 55a and the group of plasma-like spectra 55b, which are CARS spectra that have been grouped, to generate the RBC spectrum 53a and the plasma spectrum 53b respectively. At this time, a reference spectrum reflecting the characteristics of each user may be generated using the information on the group 55a of RBC-like spectra and the group 55b of plasma-like spectra obtained from the blood of an individual (user) and the standard (normal, common) RBC spectra and plasma spectra.

If, in step 76, the target spectrum has been classified as RBC-like (RBC-like spectrum) 51a, in step 77 the first analysis unit 21 performs a process of determining the glucose concentration using the RBC spectrum 53a as the analysis reference spectrum. In this process, the glucose concentration may be calculated using a preset function, or the glucose concentration may be calculated by the learning model 21a that has previously performed machine learning so as to be capable of calculating the glucose concentration. In step 76, if the target spectrum has been specified as plasma-like (plasma-like spectrum) 51b, in step 78 a process is performed to determine the glucose concentration using the plasma spectrum 53b as the analysis reference spectrum. In this process, the glucose concentration may be calculated using a preset function for spectra whose principal component is plasma, or the glucose concentration may be calculated by the learning model 21a that has previously performed machine learning so as to be capable of calculating the glucose concentration based on a reference spectrum whose principal component is plasma.

If, in step 79, it is determined that the measured glucose concentration is higher than a specified value and administration of insulin is necessary, the medication system 38 performs such administration (injection) in step 80. In addition, in step 81, the blood glucose monitor 33 may output (display) the glucose concentration in the blood via the output interface 35 and/or record the glucose concentration on an appropriate medium or in a server or the like on the cloud. In step 81, it is also possible to output the blood glucose level in the plasma (plasma blood glucose level) based on the analysis results of the plasma-like spectra 51b. In step 81, it is also possible to output the concentration of the glucose component included in other components of the blood, such as red blood cells.

As described above, when blood is used as an example of a body fluid, spectra that have been obtained in a time series by irradiating blood (body fluid) flowing through a blood vessel with laser lights will include (cyclically) repeating spectrum reflecting each of the principal components of blood, which makes it possible to obtain spectrum in which each of the principal components is classified (fractionated) according to time division (temporal sequence). In particular, since capillaries (blood vessel) under the skin that are subjected to non-invasive measurement have a small diameter and are narrow, it is easy to obtain spectra in which the components have been fractionated cyclically. By analyzing a spectrum in such spectra by referring to the spectrum that mainly reflect the principal components of blood, the influence of such principal components can be eliminated, and by referring to the peaks of the principal components, the concentrations of trace components, such as glucose, that tend to vary (fluctuate) according to biological conditions can be obtained with high accuracy.

Although obtaining the glucose concentration in blood from Raman spectra obtained by irradiating blood with one or more laser lights has been considered, if all the Raman spectra obtained from blood are averaged, the average will contain information, such as information on plasma components and red blood cells, on the principal components which have a large influence on the peaks appearing in spectra. When such spectra are averaged, large components that fluctuate over time will be averaged out, producing a large amount of noise in the information and making it difficult to detect trace components, such as glucose, that are the target of testing or measurement.

On the other hand, with the present invention, focus is placed on the fact that the CARS spectra obtained from blood flowing through blood vessels will cyclically and repeatedly include plasma-like CARS spectra and RBC-like CARS spectra, and by analyzing these separately, it is possible to prevent information on the principal components of blood from acting as noise and measure (analyze) target trace components, such as glucose, with high accuracy. That is, this measurement method has a feature of focusing on blood vessels, and in particular capillaries, which obstruct the flow of principal components of blood, such as red blood cells, and uses a blood vessel as an element that fractionates (divides, separates) the principal components of blood, which makes it possible to obtain (through time division) information (spectra) on fractions (on separation in component by component basis) of blood in which components are not uniform.

In addition, the present invention makes it possible to directly obtain the concentration of a target component in a plasma-like composition of blood, as one example, the plasma blood glucose level. It is also possible to determine a hematocrit value from the frequencies with which the plasma-like CARS spectra 51b and the RBC-like CARS spectra 51c appear.

Such processing may be performed using a learning model (AI(1)) 21a that has been trained to select plasma-like or RBC-like reference spectrum out of a plurality of CARS spectra and derive the concentration of the target component from information reflecting the target component to be measured, such as glucose, contained in plasma-like or RBC-like spectrum.

It may be possible for the reference spectra reflecting the principal components in the body fluid respectively that serve as the basis for analysis, for example, the RBC spectrum 53a and the plasma spectrum 53b, to be provided in advance. To reflect the characteristics of an individual better, the system 30 may be provided with a module (AI(2)) 22a that selects a plurality of reference spectra out of the groups 55a and 55b of a plurality of spectra in which some of the spectral components have highly similarity (correlation), within the plurality of CARS spectra 51 obtained in a time series, and thereby acquire, through self-learning, the plasma spectrum 53b indicating plasma components and the RBC spectrum 53a indicating blood cell components, for example.

Note that although blood flowing through a blood vessel has been described above as a typical example of a body fluid, it is also possible to measure components contained in other body fluids, such as lymph flowing through lymphatic vessels, in a similar manner. The “principal components” are not limited to plasma components and red blood cells, and may also include other blood cell components, such as white blood cells and/or platelets. The target component whose concentration is to be measured is not limited to glucose, and may include at least one of hemoglobin A1c, creatinine, and albumin, and/or may include any component that is measured when testing a body fluid such as blood. A favorable method for obtaining spectra that reflect a component contained in a body fluid is to use Raman scattering to obtain a scattering spectrum. Such spectra are not limited to CARS, and may be acquired using other known methods, such as stimulated Raman scattering (SRS) or surface enhanced Raman scattering (SERS). A method that acquires an absorption spectrum, such as an IR absorption spectrum, may also be used.

The above description discloses a method for detecting components of a body fluid including: irradiating a body fluid that is flowing with one or more laser lights to acquire a plurality of first spectra, which are obtained intermittently and in which the plurality of components of the body fluid are respectively reflected; and performing analysis based on one of a plurality of second spectra, in which one of the plurality of principal components of the body fluid is mainly reflected, to obtain the concentration of a target component included in the body fluid. This method may further include obtaining the plurality of second spectra from groups of a plurality of spectra having highly correlated spectral components out of a plurality of first spectra obtained in a time series. The method may further include obtaining a plurality of the second spectra from groups of a plurality of spectra that have highly correlated spectral components and periodically appear in the plurality of first spectra obtained in a time series. The method may further include self-learning the plurality of second spectra from groups of a plurality of spectra, out of the plurality of first spectra obtained in time series, in which some of the spectral components are highly correlated. A typical example of the plurality of the first spectra is Raman spectra.

The above description discloses a system that includes: a learning model that is trained to analyze spectra, which reflect components in a body fluid obtained by irradiating a body fluid with one or more laser lights, based on one out of a plurality of second spectra in which one of a plurality of principal components of the body fluid are mainly reflected and obtain a concentration of a target component included in the body fluid; and an analyzer apparatus that obtains, from a plurality of first spectra intermittently obtained by irradiating the body fluid in a flowing state with the laser light, the concentration of a target component included in the body fluid using the learning model. The system may further include an apparatus that self-learns a plurality of second spectra from groups of a plurality of spectra, out of the plurality of first spectra obtained in time series, in which some of the spectral components are highly correlated. The plurality of the first spectra may include Raman spectra.

Note that although specific embodiments of the present invention have been described above, various other embodiments and modifications will be conceivable to those of skill in the art without departing from the scope and spirit of the invention. Such other embodiments and modifications are addressed by the scope of the patent claims given below, and the present invention is defined by the scope of these patent claims.

1. A system for measuring a concentration of a component included in a body fluid, the system comprising:
an apparatus that is configured to acquire data including a plurality of spectra in a time series obtained by irradiating at least a part of the body fluid in a flowing state with laser light; and
an analyzer apparatus that is configured to analyze an analysis target spectrum, which is included in the acquired data, based on an analysis reference spectrum, which is highly similar to the analysis target spectrum and is selected out of a plurality of reference spectra that primarily reflect one out of a plurality of principal components of the body fluid respectively, and determine the concentration of a target component in the body fluid.

2. The system according to claim 1, further comprising a reference spectrum generating apparatus that is configured to determine the analysis reference spectrum based on a group of similar spectra, which include highly similar spectra that repeatedly appear among the plurality of spectra included in the acquired data.

3. A system that measures a concentration of a component included in a body fluid, the system comprising:
an apparatus that is configured to acquire data including a plurality of spectra in a time series obtained by irradiating at least part of the body fluid in a flowing state with laser light; and
an analyzer apparatus that is configured to analyze an analysis target spectrum, which is included in a group of similar spectra including highly similar spectra that repeatedly appear in the plurality of spectra included in the acquired data, based on an analysis reference spectrum including spectral components that are common to the group of similar spectra, and determine the concentration of a target component in the body fluid.

4. The system according to claim 1,
wherein the analyzer apparatus is configured to determine the concentration of the target component, which is included in any one of the plurality of principal components of the body fluid.

5. The system according to claim 1,
wherein the analyzer apparatus includes a learning model that has been trained to obtain the concentration of the target component based on the analysis reference spectrum.

6. The system according to claim 1,
wherein the apparatus that acquires the data includes a detection apparatus that is configured to acquire the data from a living organism.

7. The system according to claim 6,
wherein the detection apparatus includes a Raman spectrometer that is configured to acquire a Raman spectrum.

8. The system according to claim 6,
wherein the detection apparatus includes a probe that is configured to acquire the data on blood flowing through a blood vessel as the body fluid.

9. The system according to claim 8,
wherein the probe includes a compressor for compressing the blood vessel to control a flow rate of the blood.

10. A method of detecting a component in a body fluid, comprising:
acquiring data including a plurality of spectra in a time series obtained by irradiating a body fluid in a flowing state with laser light;
analyzing an analysis target spectrum, which is included in the acquired data, based on an analysis reference spectrum, which is highly similar to the analysis target spectrum and is selected out of a plurality of reference spectra that primarily reflect one out of a plurality of principal components of the body fluid respectively; and
determining a concentration of a target component in the body fluid.

11. The method according to claim 10,
further comprising selecting the analysis reference spectrum based on a group of highly similar spectra that repeatedly appear among the plurality of spectra included in the acquired data.

12. (canceled)

13. The method according to claim 10,
wherein the determining includes determining the concentration of the target component, which is included in any one of the plurality of principal components of the body fluid.

14. The method according to claim 10,
wherein the acquiring includes acquiring the data from a living organism.

15. The method according to claim 14,
wherein the acquiring includes acquiring a Raman spectrum.

16. The method according to claim 10,
wherein the body fluid is blood and the principal components include plasma and blood cells.

17. The method according to claim 10,
wherein the body fluid is blood, and the principal components include at least one component out of red blood cells, white blood cells, and platelets, and a plasma component.

18. The method according to claim 10,
wherein the target component includes at least one of glucose, hemoglobin A1c, creatinine, and albumin.

19. The system according to claim 3,
wherein the analyzer apparatus is configured to determine the concentration of the target component, which is included in any one of a plurality of principal components of the body fluid included in the group of similar spectra.

20. The system according to claim 3,
wherein the analyzer apparatus includes a learning model that has been trained to obtain the concentration of the target component based on the analysis reference spectrum.

21. The system according to claim 3,
wherein the apparatus that acquires the data includes a detection apparatus that is configured to acquire the data from a living organism.
